SEQUENCE LISTING (2 sequences)

SEQ ID NO: 1 - protein, 288 aa, Shewanella Oneidensis
MSSPKVGTQVADIVRKQKSSTEKNSRHKVVRHRNATTTPEMRHFIQQSELSVSQLAKILNITEATVRKWRKRESINDSPNTPHHLNTTLTPIEEYVVVGLRYQLKLPLDRLLKATQTFINPNVSRSGLARCLKRYGVSRLDEFDQELVPKQYFNQLPIAQGSDVQTYTVNSETLAKALALPSTDGDTVVQVVSLTLPPQLTEAKPKSVLLGIDSKTDWIYIDIYQDSNTQAANRYIAYVLQDGPFHLRKLLVRNYHTFLARFPGVQTPKAIAKSLNKATATRFASGDS

SEQ ID NO: 2 - DNA, 867 bp, Shewanella Oneidensis
  source 1..867: mol_type = unassigned DNA
ttatgagtctccactggcaaagcgggtggctgttgccttattaagggatttagcgatggcttttggggtttgcacgccaggaaagcgggctaaaaatgtgtggtagttgcgcaccagcaatttacgcagatgaaacggcccgtcttgcagtacataagcgatatagcgattggccgcttgggtattactgtcttgataaatatcgatataaatccaatctgtcttgctatctataccaaggagcactgatttaggcttggcttcggttagttgcggtggcagtgttaatgacaccacttgcactacggtgtcgccatcggtgcttggcagggcgagcgcttttgctaaggtttcggagttaaccgtataggtttgtacatcgctgccttgggcaatcggtaattgattaaaatactgtttagggacaagctcttggtcaaactcgtcgaggcgcgataccccataacgcttaaggcaacgggctaagcctgagcgcgatacattggggttaataaaggtttgcgtggctttaagtagcctatcgagcggcagctttagctgatacctcagtcccaccaccacatattcttcaattggggtgagggtggtattgagatgatgcggtgtattcgggctatcgttaatcgattcgcgcttacgccatttacgcactgtggcttcggtaatatttaaaattttagcaagctgactgacactcaattcagattgctgaataaagtggcgcatttctggcgtggtggtggcatttcggtgtcttaccaccttgtggcgagagtttttctctgtgctcgatttttgtttgcgaacaatatctgcgacttgagtgccaacttttggagagctcat

(19) United States
(12) Patent Application Publication
(10) Pub. No.: US 2025/0340910 A1
(43) Pub. Date: Nov. 6, 2025

(54) FATTY ACID PRODUCTION IN ESCHERICHIA COLI

(71) Applicant: University of Puerto Rico, San Juan, PR (US)

(72) Inventors: Abel Juan BAERGA ORTIZ, San Juan, PR (US); Melissa C. ORTIZ-ROSARIO, San Juan, PR (US)

(21) Appl. No.: 18/700,565

(22) PCT Filed: Oct. 20, 2022
(86) PCT No.: PCT/US2022/078424

§ 371 Date: Apr. 11, 2024

Related U.S. Application Data
(60) Provisional application No. 63/257,870, filed on Oct. 20, 2021.
(60) Provisional application No. 63/270,048, filed on Oct. 20, 2021.

(51) Int. Cl. C12P 7/6409; C12N 9/10; C12N 9/12; C12N 15/70; C12P 7/6427

(52) U.S. Cl. CPC C12P 7/6409; C12N 9/1029; C12N 9/1288; C12N 15/70; C12P 7/6427; C12Y 203/01094; C12Y 207/08007; C12N 2800/101

(57) ABSTRACT

The disclosure provides methods for increasing saturated, monounsaturated, and poly unsaturated fatty acid production using the PUFA gene cluster plasmid and the gene regulator pfaR.

### CROSS-REFERENCE TO RELATED APPLICATIONS

This application is a national stage applications under 35 U.S.C. § 371 of International Application No. PCT/US2020/078424, filed Oct. 20, 2022, which claims the benefit of priority to U.S. Provisional Patent Application No. 63/257,870, filed Oct. 20, 2021, and U.S. Provisional Application No. 63/270,048, filed Oct. 20, 2021, all of which are incorporated by reference in their entirety.

### STATEMENT REGARDING FEDERALLY SPONSORED RESEARCH

This invention was made with government support under award numbers CHE0953254 awarded by the National Science Foundation and R25M061838 awarded by the National Institute of Health. The government has certain rights in the invention.

### REFERENCE TO AN ELECTRONIC SEQUENCE LISTING

This application contains a Sequence Listing that has been submitted electronically and is hereby incorporated by reference in its entirety. The Sequence Listing was created on May 12, 2025, is named “21-1436-WO-US Sequence Listing.xml”, and is 3,935 bytes in size.

### FIELD OF THE INVENTION

The present disclosure relates to biosynthesis of fatty acids. More specifically, the disclosure relates to use of the transcriptional regulator of eicosapentaenoic acid synthesis (pfaR) to enhance biosynthesis of fatty acids in E. coli.

### BACKGROUND

Fatty acid (FA) biosynthesis in bacterial and microbial cultures offers a promising solution to sustainable generation of biofuels and biomaterials. For example, FA production in E. coli has the advantages of low environmental impact, short production times, and ease of genetic manipulation. Fatty acids are also widely used as nutritional supplements and as biofuels, two areas that have driven a search for efficient and sustainable methods for their production¹⁴⁻¹⁷. This is especially true for poly unsaturated fatty acids (PUFAs). Consistent with this, free fatty acids produced in the metabolic pathways of bacteria such as Escherichia coli (E. coli) can be used, for instance, as precursors for biofuels (Magnuson, K., et al. J Regulation of fatty acid biosynthesis in Escherichia coli. Microbiol. Rev. 57, 522-542 (1993).

FA biosynthesis is a ubiquitous pathway across organisms of varied phylogenies. Specifically, FA synthesis requires concerted action by multiple enzymes that catalyze condensation of acyl units. Consistent with this, polyunsaturated fatty acids (PUFA) are naturally made in anerobic environment, including deep-sea bacteria. Additionally, select species of marine bacteria are capable of omega-3 fatty acid (ω3-FA) production through use of the polyunsaturated fatty acid (PUFA) synthase pathway, an enzyme pathway for secondary metabolism. PUFA synthases achieve de novo synthesis of specific long-chain PUFA using the corresponding double bond pattern from acetyl-CoA units

PUFA synthases hold significant industrial promise if production could be scaled. Escherichia coli (E. Coli), is an organism for which a myriad of genetic tools are accessible and whose metabolism is well described. Further since wild type E. coli does not produce PUFAs, they are a model system for observing effects of introducing and deleting PUFA genes, on the production of specific fatty acids. Consistent with the, the transfer of PUFA genes into E. Coli has resulted in identification of five genes critical for PUFA production (FIG. 1, Type I), which are pfaA, pfaB, pfac, pfaD and pfaE.

There remains a need in the art for enhanced FA biosynthesis using the pfaA, pfaB, pfac, pfaD and pfaE PUFA synthases. For examples, mechanisms that exploit these synthases to drive PUFA synthesis on an industrial scale.

### SUMMARY

The present disclosure provides a method for increasing fatty acid production using the pEPA Δ1,3,4,9 gene cluster in conjunction with the pfaR over expression in E. coli unable to naturally produce polyunsaturated fatty acids.

In one aspect is an in vitro method of producing fatty acids, wherein an E. coli containing pfaA, pfaB, pfaC, pfaD and/or pfaE genes (pEPAΔ1, 3, 4, 9) overexpresses gene regulator pfaR.

In one embodiment of the first aspect, the E. coli strain is JM109.

In one embodiment of the first aspect the E. coli strain is DH10B.

In one embodiment of the first aspect the E. coli is incubated at 15° C.

In one embodiment of the first aspect the E. coli are grown in cell culture flasks, tubes, or bags containing media and nutrients equivalent to twenty percent of the total volume.

In one embodiment of the first aspect E. coli are grown in Laura Broth without carbon supplementation.

In one embodiment of the first aspect endogenous acyl-CoA synthetase structural gene, fadD is deleted.

In one embodiment of the first aspect saturated, monosaturated and polyunsaturated fatty acid synthesis is increased.

In one embodiment of the first aspect the increased polyunsaturated fatty acid increase includes omega-3 three and omega 6 fatty acids.

In a second aspect is a pfaRso plasmid, wherein the gene sequence of pfaRso has greater than 90% homology to SEQ ID NO. 1.

In one embodiment of the second aspect the plasmid is grown under anaerobic conditions.

In one embodiment of the second aspect the plasmid contains pfaA, pfaB, pfaC, pfaD and/or pfaE genes (pEPAΔ1, 3, 4, 9).

### BRIEF DESCRIPTIONS OF THE DRAWINGS

FIG. 1. Pfa gene cluster. (FIG. 1A) The pfa gene cluster from Shewanella oneidensis and its encoded multidomain protein products are well conserved across a range of microbial organisms. (FIG. 1B) The pfaR gene lies directly downstream of the pfaA gene when it is present. However, some pfa clusters lack a pfaR gene.

FIG. 2. Gas chromatogram showing identities of each peak after full mass spectrum analysis of each peak against NIST MS search 2.0. (FIG. 2A) E. coli DH5α pEPA Δ1,3,4,9, (FIG. 2B) E. coli DH5α pfaR, and (FIG. 2C) E. coli DH5α pEPA Δ1,3,4,9 pfaR. None of the conditions produced polyunsaturated fatty acids.

FIG. 3. Fatty acid profile for E. coli. (FIG. 3A) Fatty acid profile of E. coli DH10B grown at 15° C. Some strains carrying the pEPAΔ1,3,4,9 plasmid produced a 20-carbon chain length fatty acid but were while unable to produce polyunsaturated fatty acids. (FIG. 2B) Fatty acid profile of E. coli DH10B grown at 20° C. Although there are differences in the fatty acid profile when comparing the WT versus the recombinant strain there is neither production of long chain fatty acids nor polyunsaturated fatty acids.

FIG. 4. Fatty acid production in E. coli DH10B with increased headspace in different vessels. Fatty acid profile of E. coli DH10B transformed with pEPAΔ1,3,4,9 grown on different vessels. The condition in which production of PUFAS, including EPA, was achieved is the culture grown in the 100 mL vessel. (3^{rd} bar from left in each grouping).

FIG. 5. E. coli DH10B pEPAΔ1,3,4,9 fatty acid profile with and without glucose or glycerol supplementation in comparison to the control E. coli DH10B pET100. PUFA production was achieved with the pEPAΔ1,3,4,9 strain in just one of three replicates (FIGS. 5A-5C).

FIG. 6. Functional cassettes. (FIG. 6A) Functional cassette for recombination with homology arms (highlighted yellow sequence) introduced by PCR using primers with overlapping sequences (primer sequences include the sequences inside the boxes and yellow highlighted sequences). The sequence in red letters is the gene that confers antibiotic resistance (neoR). (FIG. 6B) Verification through agarose gel electrophoresis of the functional cassette which band (pointed with orange arrow) was used for excision and purification.

(Lane a) MW ladder, (Lanes b-c) functional cassette, (Lane d) negative control. (FIG. 6C) Recombination reaction with the cassette designed to have arms homologous to sequences in the gene of interest which results in its substitution with the antibiotic resistance marker.

FIG. 7. Fad Sequence. (FIG. 7A) The fadD sequence. Portions in between the highlighted yellow sequences are replaced by portions in between the yellow highlighted sequences of the functional cassette in FIG. 6A. (FIG. 7B) Deletion verification. The orange arrow indicates the expected size of the amplicon. (Lane a) MW ladder, (Lanes b-e) mutants carrying the deletion, (Lanes f-i) negative controls, (Lane j) positive control

FIG. 8. Fatty Acid Production in E. coli JM109. Fatty acid profile of E. coli JM109 grown at 15° C. Only pEPA Δ1,3,4,9 and pEPA Δ1,3,4,9-pfaR carrying strains show PUFA production (16:4, 18:2, 18:4, 20:3, 20:4, 20:5, 22:2, 22:4, 22:5). The only PUFA showing a decrease in pEPA Δ1,3,4,9-pfaR relative to pEPA Δ1,3,4,9 is 20:5 (eicosapentaenoic acid) while all other PUFAs show increase concentrations in pEPA Δ1,3,4,9-pfaR. Almost all monounsaturated FA are increased in pEPA Δ1,3,4,9 and pEPA Δ1,3,4,9-pfaR carrying E. coli, the only exceptions are 16:1 and 17:1. Saturated FAs do not follow the same pattern in pEPA Δ1,3,4,9 and pEPA Δ1,3,4,9-pfaR.

FIG. 9. Summary for E. coli JM109 FA Profile. Fatty acid profile of E. coli JM109 grown at 15° C. summarizing FA distribution seen in FIG. 8 by type. As shown in FIG. 8 pET-pEPAΔ1,3,4,9 and pfaR-pEPAΔ1,3,4,9 show increased levels of monounsaturated and polyunsaturated FAs while saturated FAs stay comparable to the controls for pET-pEPAΔ1,3,4,9 but not for pEPAΔ1,3,4,9-pfaR. There is a significant difference in the total FAs produced in pfaR-pEPAΔ1,3,4,9 when compared to pET-pEPAΔ1,3,4,9 in which main driver is the monounsaturated FA yield and the saturated FA yield also is a contributor to a lesser extent.

### DETAILED DESCRIPTION

Unless defined otherwise, all technical and scientific terms used herein have the same meaning as is commonly understood by one of ordinary skill in the art. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). These references are intended to be exemplary and illustrative and not limiting as to the source of information known to the worker of ordinary skill in this art. As used herein, the following terms have the meanings ascribed to them below, unless specified otherwise.

It is noted here that as used in this specification and the appended claims, the singular forms “a,” “an,” and “the” also include plural reference, unless the context clarity dictates otherwise.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As utilized in accordance with the present disclosure, the following terms, unless otherwise indicated, shall be understood to have the following meanings. Unless otherwise required by context, singular terms shall include pluralities and plural terms shall include the singular.

“Fatty Acid (FA) biosynthesis” as used herein refers to a ubiquitous pathway in almost all prokaryotic and eukaryotic organisms. FA biosynthesis in bacterial and microbial cultures provides a unique solution to sustainable generation of biofuels and biomaterials as well as human nutrition. Although FA biosynthesis can be carried out in a wide range of organisms, E. coli offers advantages of low environmental impact, short production times, and ease of genetic manipulation.

E. Coli FA biosynthesis takes place through action of a dissociated system in which several different enzymes catalyze a series of chemical steps for the condensation of acyl units. Each protein is encoded by a separate gene and as such, in E. coli, the Fab enzymes, as well as ACP, are separate domains that act as independent entities. While E. coli is the preferred microorganism for FA biosynthesis, one skilled in the art will understand that the dissociated system is common among other bacteria, plants, and algae, such as, but not limited to Yarrowia Lipolitica (a yeast), Synechococcus elongatus (a cyanobacteria) and Thraustochytrids (marine protists). (Ohlrogge, J. B. & Jaworski, J. G. Regulation of fatty acid synthesis. Annu. Rev. Plant Physiol. Plant Mol. Biol. 48, 109-136 (1997); Rock, C.O. & Jackowski, S. Forty years of bacterial fatty acid synthesis. Biochem. Biophys. Res. Commun. 292, 1155-1166 (2002). As such, one skilled in the art will understand that the biosynthesis of FA described can also be achieved in a wide range of additional microorganisms, including but not limited to other bacteria, plants, and algae. One of skill in the art will know this further includes, but is not limited to, Yarrowia Lipolitica (a yeast), Synechococcus elongatus (a cyanobacteria) and Thraustochytrids (marine protists).

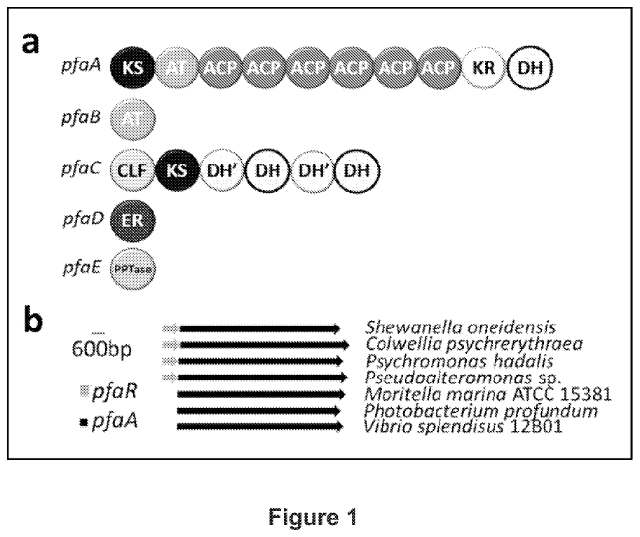

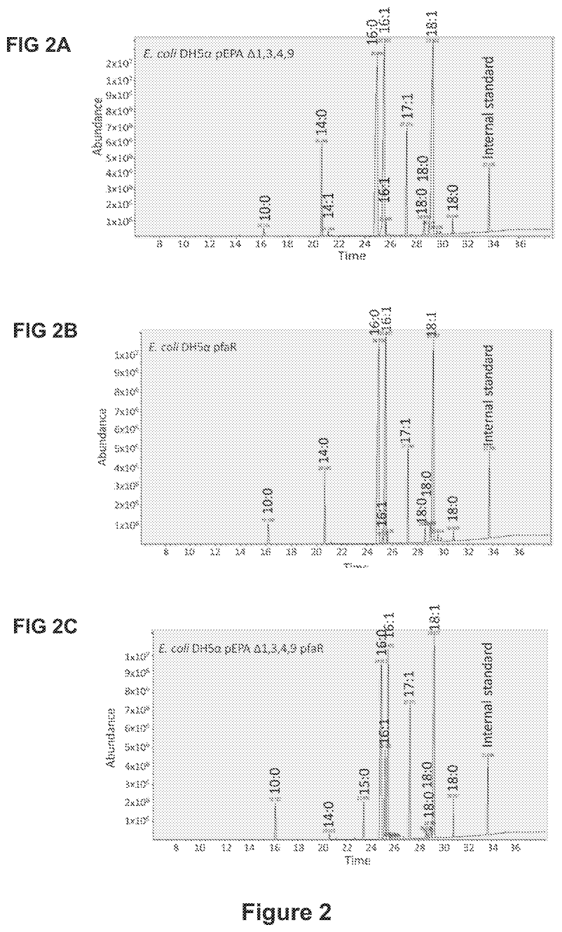

Each FA elongation cycle consists of four reactions required to extend the length of the acyl chain by two carbons (FIG. 1). Initiation of FA synthesis requires the carboxylation of acetyl-CoA to produce malonyl-CoA which is then condensed with an acetyl-CoA by a Claisen condensation catalyzed by FabH to generate β-ketobutyryl-ACP.

“Polyunsaturated fatty acid synthases” as used herein refers to Type I iterative enzymes that are responsible for carrying out de novo synthesis of long-chain PUFAs. Malonyl CoA is used as the carbon source and the products include a numerous fatty acid, having two or more double bonds. One of skill in the art will understand that exemplary polyunsaturated fatty acids, include but are not limited to Docosahexaenoic acid (22:6; n-3); Docosaspentaenoic acid (22:5; n-6); Eicosapentaenoic acid (20:5; n-3); and Arachidonic acid (20:4; n-6), or combinations of PUFAs. PUFAs are unique in that they do not require molecular oxygen to form carbon-carbon double bonds. This contrasts with formation of PUFAs using elongase and desaturase reactions from short-chain fatty acids.

“PUFA gene cluster”, as used herein, refers to the cluster of synthases involved in the formation of PUFAs. The PUFA gene cluster is comprised of multiple subunit combinations of pfaA, pfaB, pfac, pfaD and pfaE. One of skill in the art will also understand that additional genes critical for poly unsaturated fatty acid synthesis may be part of the gene cluster.

Transcriptional regulator of eicosapentaenoic acid synthesis” (pfaR) refers to a regulator of the PUFA synthase gene cluster. The inventors of the current disclosure have identified that overexpression of pfaR in conjunction with the PUFA gene cluster can be used to artificially increase production of fatty acids.

“pEPAΔ1, 3, 4, 9” refers to the plasmid containing the minimum set of genes required for Type I PUFA production.

“pfaR-pEPAΔ1, 3, 4, 9” as used herein refers to expression of the pfaR gene in E. coli using the pEPA plasmid containing the pEPAΔ1, 3, 4, 9 pfa gene cluster, from Shewanella pneumatophori. The control plasmid as used herein can be either the pfaR regulatory gene or the pEPAΔ1, 3, 4, 9 plasmid. The regulator pfaR, as demonstrated herein, has minimal impact on the production of EPA or DHA but likely contributes or dives the increase in the total PUFA yields when expressed in large amounts. Bioinformatic analysis of the pfaR sequence, which includes the analysis of the sequence conservation and its location within the cluster, suggests that pfaR has a relevant function in PUFA synthesis as well as a regulatory role.

The current disclosure describes the use of pfaR to regulate or enhance the PUFA synthase machinery. The use of pfaR as a regulator as described herein results in a surprising technical effect wherein PUFA synthesis is increased over previous attempts to increase PUFA synthesis, as summarized in Table 1 wherein similar attempts resulted in different degrees of success in producing different amounts of EPA or docosahexaenoic acid (DHA) in E. coli as shown in Table 1.

“Aeration” as used herein refers to the head space in the cell culture flask. The variety of the shapes of the cultivation vessels gives rise to differences in the head-space of the culture which in turn affects the aeration, oxygenation and shear stress conditions experienced by the bacteria.¹⁰

“Fatty Acids” as used herein refer to a carboxylic acid with an aliphatic chain, which is either saturated or unsaturated. The term “saturation” refers to the degree of hydrocarbon saturation with hydrogen bonds. It is understood that mono- and poly-unsaturated fatty acids have one or more double bonds with a terminal carboxylic group. One of skill in the art will understand that classes of fatty acids include unsaturated, monounsaturated, and polyunsaturated fatty acids and that numerous fatty acids exist in each class.

“pfaRso” as used herein refers to the plasmid developed by the inventors of the present disclosure containing the Transcriptional regulator of eicosapentaenoic acid synthesis PfaR. The backbone was pET200 and the protein and gene sequence provided in Table 5 from Shewanella Oneidensis, an advantageous bacterium for its ability to live in aerobic or anaerobic environments.

### EXAMPLES

The Examples that follow are illustrative of specific embodiments of the disclosure, and various uses thereof. They are set forth for explanatory purposes only and should not be construed as limiting the scope of the disclosure in any way.

### Materials Used in Examples

Solutions and equipment used in the following Examples are briefly set forth herein.

E. coli Cells

E. coli JM109 competent cells were purchased from Promega. E. coli DH5α and DH10B and plasmid pET200 were purchased from ThermoFisher. Plasmid pEPAΔ1,3,4,9 was obtained from a colleague and plasmid pWE15 obtained from ATCC. The plasmid pfaRso was created by the inventors with the pfaR gene having the amino acid sequence of SEQ ID No. 1.

Reagents including kanamycin, ampicilin, LB broth, acetyl chloride, methanol, hexane, chloroform, methyl heneicosanoate and BHT were purchased from Sigma.

### Bacterial Transformation and Growth

E. coli JM109 competent cells were transformed with pPfaRso, pEPAΔ1,3,4,9,pWE15, and/or pET200, by electroporation. The transformants were picked by antibiotic selection (Laura Broth (LB) agar with KAN 50 μg/ml, AMP 100 μg/ml or both) and used to prepare a 20 ml pre-culture in LB maintaining antibiotic selection as set forth above. After 72 hours of growth shaking at 160 rpm at 30° C., 8 ml of culture was seeded for an 800 ml culture in LB maintaining antibiotic selection as set forth above. The culture was grown at 160 rpm at 15° C. for 96 hours.

Media for Supplementation with Glucose and Glycerol

The media used for aeration experiments was LB broth with appropriate antibiotic (LB agar with KAN 50 μg/ml, AMP 100 μg/ml or both) depending on the plasmid(s) used. LB broth was supplemented with glucose to a final concentration of 22.2 mM in LB broth while supplementation of glycerol consisted of a final glycerol concentration of 22 mM.

### Flasks for Aeration Experiments

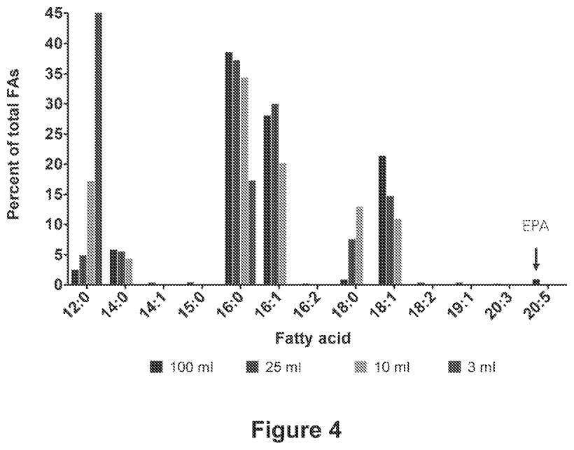

The flasks used for the aeration experiments were contained 3 ml of broth for a 15 ml conical tube; 10 ml of broth for a 50 ml conical tube; 25 ml of broth a 125 ml Erlenmeyer flask; and 100 ml of broth for a 500 ml Erlenmeyer flask. The variety of the shapes of the cultivation vessels gives rise to differences in the head-space of the culture which in turn affects the aeration, oxygenation and shear stress conditions experienced by the bacteria¹⁰. As noted herein the four different flasks (15-and 50-ml conical tubes and 125 and 500 ml Erlenmeyer flasks) were filled to ⅕th their total volume capacity to evaluate whether it was possible to obtain EPA under any of these conditions using the DH10B strain transformed with pEPAΔ1,3,4,9. Results demonstrate EPA production in the 500 ml flask filled to ⅕th its total volume capacity and grown at 15° C. as shown in FIG. 4.

### FadD Deletion

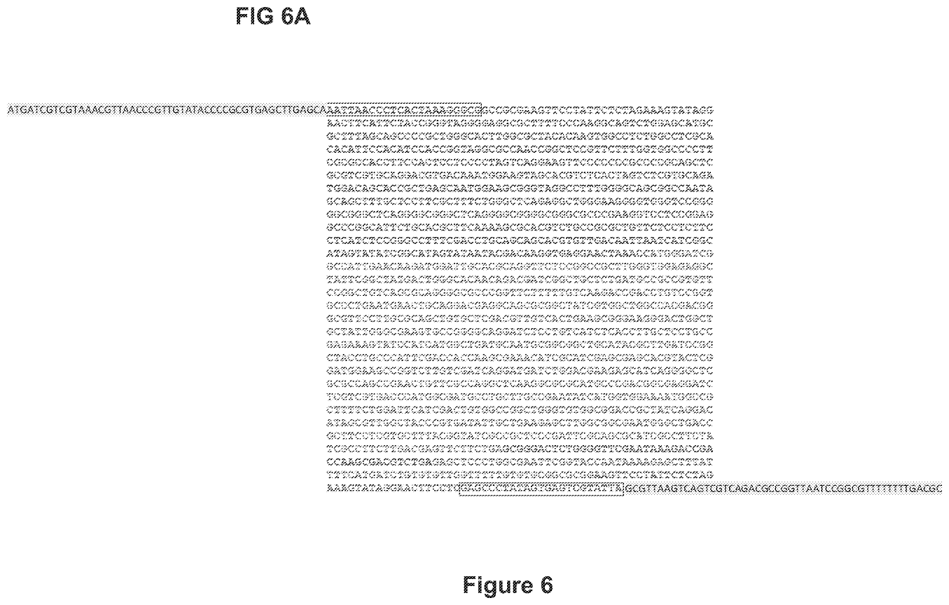

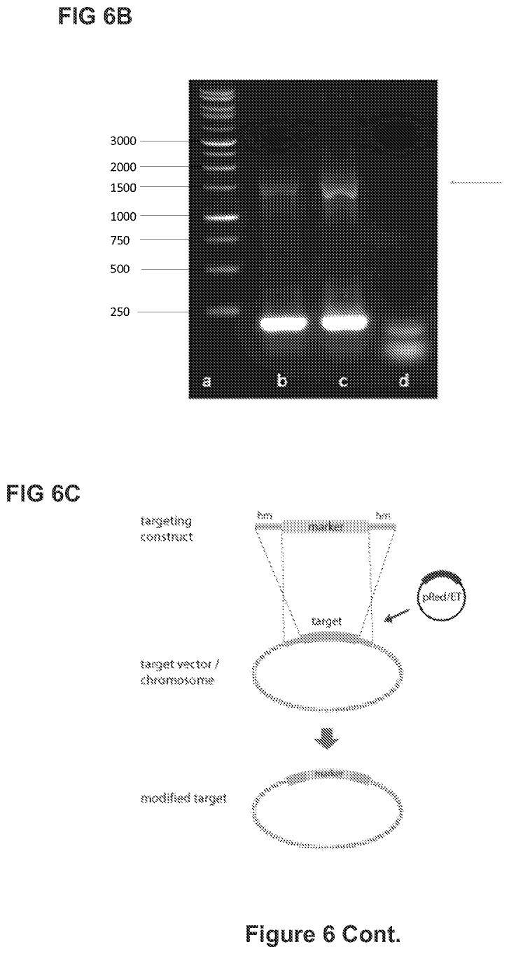

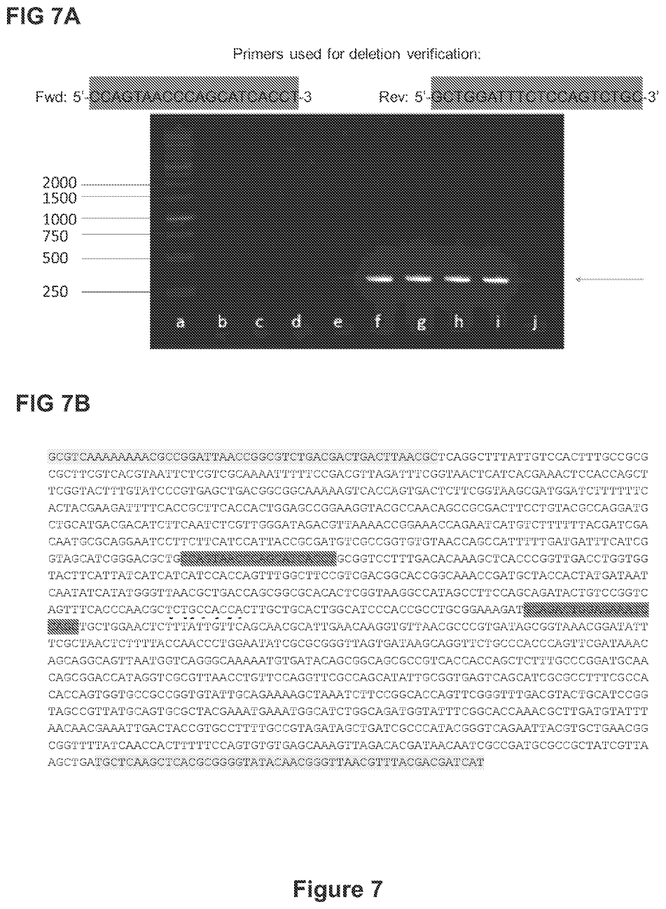

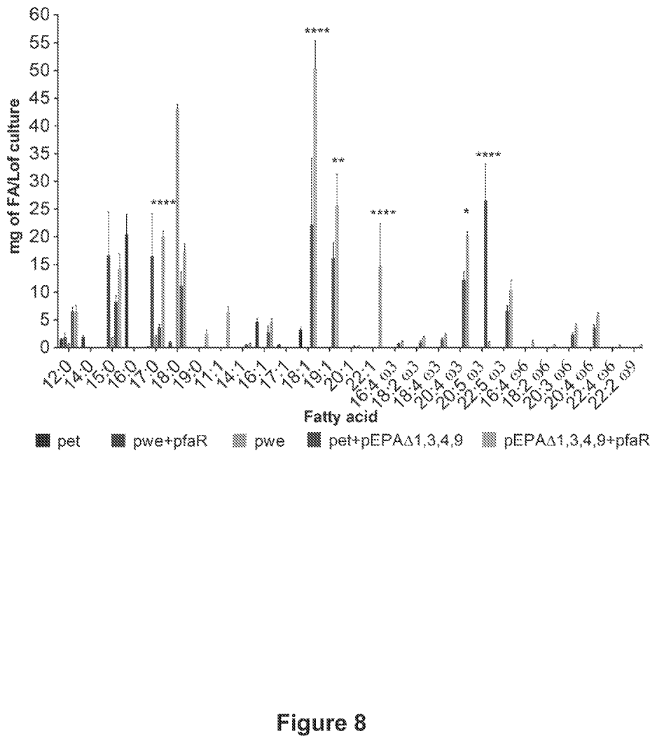

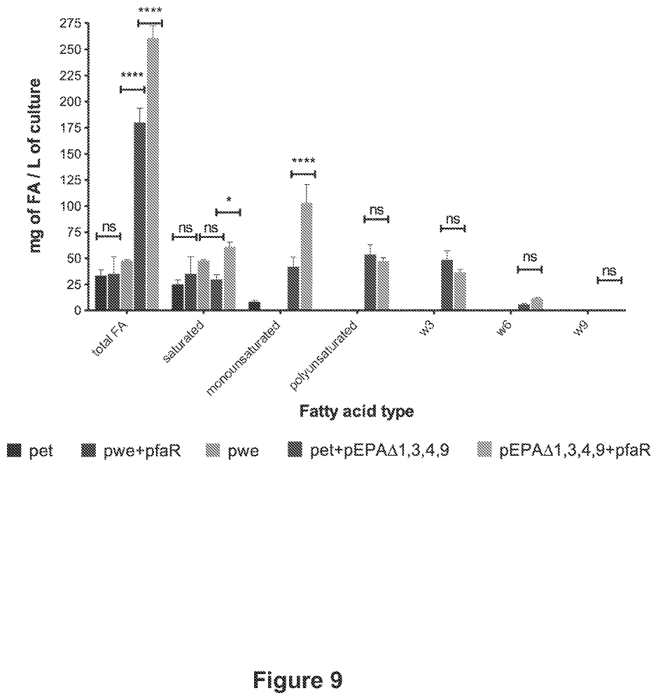

FadD deletion was performed using GeneBridges Red/ET Quick & Easy E. coli Gene Deletion Kit (see FIG. 6). Briefly, a functional cassette with homologous arms was generated by PCR amplification, followed by gel electrophoresis (see FIG. 6B), extraction of the band and purification of the DNA product. E. coli cells were transformed with the pRedET plasmid, which codes for the recombinase, by electroporation. Recombinant strains were picked by antibiotic selection using LB agar plates with 50 μg/ml ampicillin. Recombinant strains were grown in LB broth with 50 μg/ml ampicillin and 0.3% L-arabinose to induce recombinase expression. The functional cassette was introduced by electroporation and colonies carrying the deletion were selected by steaking on a LB plate containing kanamycin (15 μg/ml). Deletion was confirmed by PCR amplification of part of the target gene (FIG. 7A).

### Cell Harvesting and Fatty Acid Extraction

Bacterial cells were harvested by centrifugation at a speed of 5,000 g, at 4° C. for 15 min. Pellets were freeze-dried, and material used for fatty acid extraction using the Bligh-Dyer method followed by acid-catalyzed methanolysis. During this process, methyl heneicosanoate was added as an internal standard, and BHT was added to avoid sample oxidation before analysis. After this process, the sample was completely dried under a nitrogen stream and resuspended in hexane for GC-MS analysis.

### GC-MS Analysis

Fatty acid profiling was performed using a GC/MS-QP2010 (Shimadzu) equipped with a fused-silica FAMEWAX capillary column (Restek, PN 221-75940-30, 30 m×0.32 mm i.d.×0.25 μm film thickness) and an auto-injector (Shimadzu, AOC-20i). Samples were injected (injection volume 1 μl) in the split mode (split ratio=15:1, liner split line PN 220-90784-00). The oven temperature was set to 130° C. for 5 min, followed by a temperature ramp from 130° C. to 250° C. at 4° C./min, and held at 250° C. for 5 min, using helium as the carrier gas at a flow rate of 1 ml/min. Mass spectra data were obtained after electron impact ionization (Detector: EI, 70 eV, ion source temperature 200° C.) in full scan mode between 50 and 600 amu. Injection port and detector temperatures were set at 250° C.

Identification of mass spectrum peaks was carried out with using GCMSsolution software (Shimadzu) with the NIST 14 library while also comparing retention times with the Supelco 37 FAME mix external standard³¹. Quantitation of each fatty acid was performed by comparison of the area below the peak of the peak of interest versus the area below the peak of the internal standard³²

### Example 1. Strain Selection

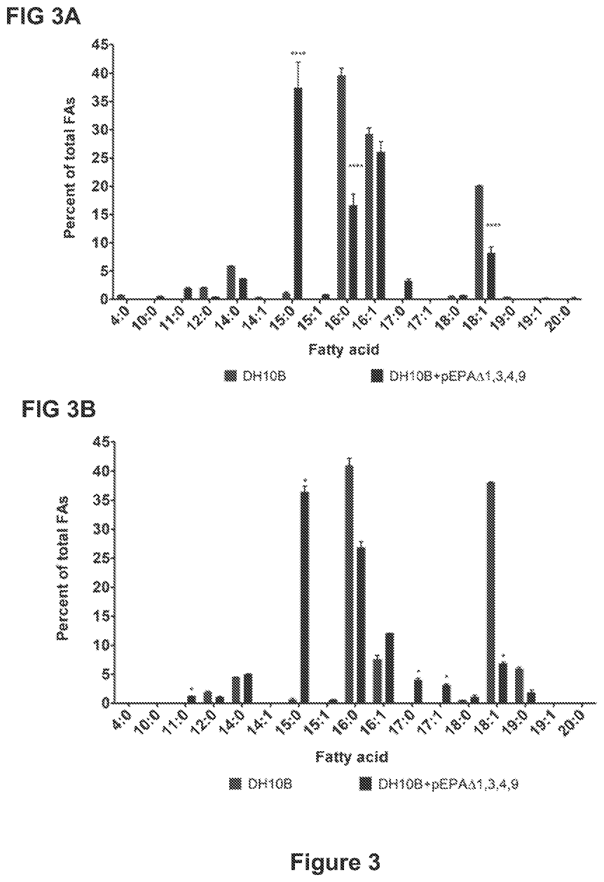

Initial attempts to study the effects of a pfaR on the fatty acid profile of E. coli were made using the DH5α strain. Strains harboring the pEPAΔ1,3,4,9 plasmid did not produce eicosapentaenoic acid (EPA) or any other PUFA when grown at 15° C. FA profiles for the strain harboring either PfaR by itself or in conjunction with pEPAΔ1,3,4,9 were similar to the FA profile obtained or the pEPAΔ1,3,4,9 strain, both of which showed no PUFA production (FIG. 3).

The DH10B strain was chosen for PUFA production because of its genotypic similarity to DH5α. Experiments were performed at 15° C. and 20° C. (consistent with prior art); temperatures previously reported to be associated with EPA and DHA production in E. coli (See Table 1).

TABLE 1
EPA or DHA production in E. coli ¹¹

| | | | | Growth | Time |
|---|---|---|---|---|---|
| Strain | Gene Source | Product | Amount | Temp (° C.) | (h) |
| E. coli | S. putrefaciens | EPA | 3.3 mg/g | 20 | ND |
| S17-1 | SCRC-2738 | | DCW | | |
| E. coli | S. putrefaciens | EPA | 5.7% | 20 | ND |
| JM109 | SCRC-2738 | | | | |
| E. coli | S. pneumatophori | EPA | 22% | 15 | ND |
| JM109 | SCRC-2738 | | | | |
| E. coli | Shewanella | EPA | 0.7% | 20 | 34 |
| XL1-Blue | oneidensis MR-1 | | | | |
| E. coli | Shewanella | EPA | 7.5% | 20 | 36 |
| | oneidensis MR-1 | | | | |
| E. coli | Moritella marina | DHA | 5.2% | 15 | 96 |
| DH5α | MP-1 | | | | |
| E. coli | Shewanella sp. | EPA | 12% | 20 | 40 |
| DH5α | Strain SCRC 2738 | | | | |
| E. coli | M. marina MP-1 and | DHA | 0.2% | 15 | 96 |
| DH5α | S. pneumatophori | | | | |
| | SCRC-2738 | | | | |
| E. coli | M. marina MP-1 and | EPA | 9.2% | 15 | 96 |
| DH5α | S. pneumatophori | | | | |
| | SCRC-2738 | | | | |
| E. coli | S. baltica MAC1 | EPA | 14% | 15 | 120 |
| EPI300T1 | | | | | |
| E. coli | S. baltica MAC1 | DHA | 0.4% | 15 | 120 |
| EPI300T2 | | | | | |
| E. coli | S. baltica MAC1 | EPA | 31.4 mg/g | 15 | ND |
| Nissle1917 | | | DCW | | |
| E. coli | C. psycherythraea | DHA | 2.2 mg/g | 15 | ND |
| DH5α | 34H and S. | | DCW | | |
| | baltica MAC1 | | | | |

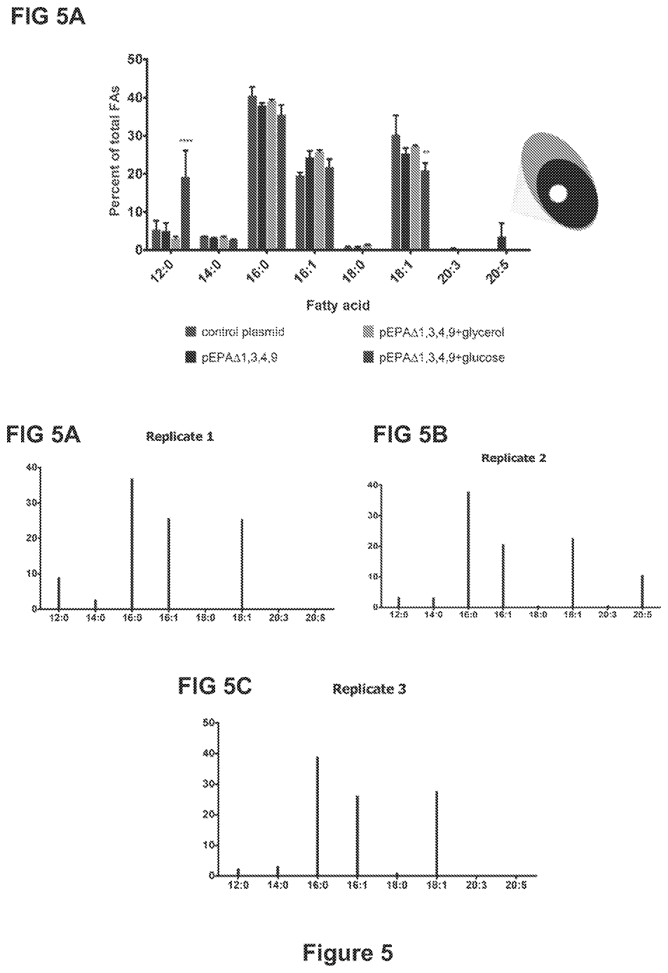

Unexpectedly, and as show in FIGS. 5 and 6 EPA and other PUFAs were not produced at 15° C. and 20° C. in the DH10B strain.

Unexpectedly, a PUFA with a 20-carbon chain length at the 15° C. condition was produced on one of the pEPA replicates. This uncharacteristic 20-carbon product is attributable to the presence of the PUFA muti-enzymes, since the product is not present in the control and PUFA synthase is known to be specific for C20 or C22 carbon chain lengths. As a result experiments were performed at 15° C.

### Example 2. Aeration

The variety of the shapes of the cultivation vessels gave rise to differences in the head-space of the culture which in turn affected the aeration, oxygenation and shear stress conditions experienced by the bacteria¹⁰. Accordingly, four different flasks (15- and 50-ml conical tubes and 125 and 500 ml Erlenmeyer flasks) were filled to ⅕^{th} their total volumetrice capacity to evaluate whether it was possible to obtain EPA under any of these conditions using the DH10B strain transformed with pEPAΔ1,3,4,9. EPA production was attained in the 500 ml flask filled to ⅕^{th} its total volume capacity and incubated at 15° C., as shown in FIG. 4.

### Glucose Supplementation and Culture Metabolism

Fatty acid productions in bacteria can be dependent on glucose or other carbon source supplementation. Initial experiments utilized standard E. coli culture media without carbon supplementation. E. coli strain DH10B resulted in PUFA production, including EPA, in the pEPAΔ1,3,4,9 strains grown in LB without further supplementation (FIG. 5).

Supplementation with glucose did not result in PUFA production in strains harboring the pEPAΔ1,3,4,9 plasmid. Increases in dodecanoic acid (12:0) and a decrease in 9-octadecenoic acid (18:1; FIG. 5) were present under these conditions. Supplementation with glycerol for the same clone did not produce any PUFAs or result in any remarkable change in the FA profile of the E. coli. As a result E. coli DH10B was selected as an adequate candidate for PUFA synthesis using LB without further supplementation.

### Example 3: Accumulation of FAs through Deletion of FadD

In order to reduce the possibility of FA degradation, resulting in an absence of PUFA, and to cause a maximum of fatty acid accumulation, the endogenous fadD of E. coli, was deleted. The endogenous fadD encodes the long chain FA CoA ligase that converts FAs into metabolically active CoA thioesters committed to degradation. Deletion of fadD was achieved with the Red/ET recombination method, producing clones resistant to either neomycin or kanamycin (by way having neoR insertion in place of fadD).

A functional cassette having arms homologous to flanking sequences of fadD was created using PCR, sequences used for this purpose are shown in FIG. 5. The product was then excised from the gel, solubilized, and purified and used in conjunction with a plasmid containing the sequence coding for the recombinase to transform E. coli followed by expression of the recombinase by the addition of arabinose. Successful deletion of fadD was confirmed by agarose gel electrophoresis (FIG. 7). Individual mutant colonies that had been grown on solid LB agar media with antibiotics, confirmed to be carrying the fadD deletion, were selected to grow in liquid LB media with antibiotics.

### Example 4: JM109 E. coli Strain

Replicate experiments were performed, as described using the JM109 strain which prior art teaches is a strain that produces a greater proportion of EPA when harboring the entire PUFA gene cluster but is not associated with the production of other fatty acids¹¹⁻¹³.

JM109 was grown in two-liter Erlenmeyer flasks at 15° C. for 96 hours using LB media. An unexpected and surprising technical effect was EPA production in the JM109 strain.

Analysis of FA production demonstrated expression of pEPAΔ1,3,4,9 in E. coli JM109 resulted in a four-fold increase in total FA production when compared to control. The increase was related to production of monosaturated and polyunsaturated FAs whereas surprisingly, the production of saturated FA did not change significantly. A surprising technical effect, monounsaturated FAs demonstrated a seven-fold increase vs the control experiments, while PUFAS were not detectable in the control to attributing to 43% of the total FA produced in the presence of pEPAΔ1,3,4,9. In addition, ω3-FAs besides EPA were produced as shown in Table 2 and w6-fatty acids as shown in Table 3.

TABLE 2
Non-EPA, ω3-Fatty Acids produced in JM109 E. coli

| | |
|---|---|
| Methyl 4,7,10,13-hexadecatetraenoate | (16:4 ω3) |
| 12,15-Octadecadienoic acid, methyl ester | (18:2 ω3) |
| Methyl stearidonate | (18:4 ω3) |
| Methyl 5,11,14,17-eicosatetraenoate | (20:4 ω3) |
| Methyl 8,11,14,17-eicosatetraenoate | (20:4 ω3) |
| Methyl 7,10,13,16,19-docosapentaenoate | (22:5 ω3) |

TABLE 3
ω6-Fatty Acids produced in JM109 E. coli

| | |
|---|---|
| Methyl 7,11,14-eicosatrienoate | (20:3 ω6) |
| 8,11,14-Eicosatrienoic acid, methyl ester | (20:3 ω6) |
| 5,8,11,14-Eicosatetraenoic acid, methyl ester | (20:4 ω6) |

### Example 5: pfaR Expression in E. coli JM109

Expression of pfaR in E. coli JM109 was achieved by transformation with the pfaR containing plasmid in conjunction with pWE15 plasmid. The bacterial cultures were grown at 15° C. and 160 rpm for 96 hours. Expression of pfaR resulted in the absence of monounsaturated FAs when compared to the control carrying pET200 and reduction of total FA yield including an absence of PUFAs.

Co-Expression of pfaR with pEPAΔ1,3,4,9

Co-expression of pfaR-pEPAΔ1,3,4,9 resulted in an unexpected five-fold increase in the total FA yield when compared with the controls not carrying pEPAΔ1,3,4,9. This increase was observed in saturated, mono and polyunsaturated FAs. A two-fold change when compared to pET-pEPAΔ1,3,4,9 without pfaR. This change was associated in large-part to increases in saturated and monosaturated FAs. Expression changes in total FAs between pET-pEPAΔ1,3,4,9 and pEPAΔ1,3,4,9-pfaR were related to increased production of FAs 17:0, 18:1, 19:1, 22:1, and 20:403. In addition, a decrease in 20:5ω3 (EPA) was also observed. In a further surprising, unexpected technical effect, the 19:0, 11:1, 22:1, 16:4006, 18:206, 22:406 and 22:209 fatty acids were uniquely produced by the clones carrying pEPA Δ1,3,4,9-pfaR.

The combinations of pET-pEPAΔ1,3,4,9 and pfaR-pEPAΔ1,3,4,9 are unique in that the monounsaturated FA exceed saturated FA content and the production of PUFAs including omega-3 fatty acids produced by the strains as shown in Table 4.

TABLE 4
Omega 3 Fatty Acids Produced in combinations of
pET-pEPAΔ1,3,4,9 and pfaR-pEPAΔ1,3,4,9

| | |
|---|---|
| Methyl 4,7,10,13-hexadecatetraenoate | (16:4ω3) |
| 12,15-Octadecadienoic acid, methyl ester | (18:2 ω3) |
| Methyl stearidonate | (18:4 ω3) |
| Methyl 5,11,14,17-eicosatetraenoate | (20:4 ω3)* |
| Methyl 8,11,14,17-eicosatetraenoate | (20:4 ω3)* |
| Methyl eicosa-5,8,11,14,17-pentaenoate | (20:5 ω3)* |
| Methyl 7,10,13,16,19-docosapentaenoate | (22:5 ω3) |

(*p < 0.05)

### Example 6: Fatty Acid Production in E. Coli

Aeration and ω3-FA production

E. coli strains DH5α and DH10B, both of which are E. coli K12 derivatives with similar genotypes, are sensitive to aeration, which increases with culture head-space¹⁸, thereby serving as a factor for ω3-FA production in these E. coli strains,

### Deletion of Fatty Acid Degrading Enzymes and Strain Selection

Deletion of the fatty acid-degrading enzyme encoded by the fadD gene, long chain FA-CoA-ligase, of E. coli in strain DH10B produced a genotype that when transformed with pEPA was able to grow in solid media but surprisingly, not in a liquid media that contained the same nutrients.

E. coli strains DH5α and DH10B are known in the art to be associated with problematic replication. This variable does not seem to affect E. coli strain JM109 probably having to do with its proline auxothrophy (inability to synthesize proline)^{20,21}. The inability to synthesize proline in this stain arises specifically from the mutations in proA and proB genes that are part of the metabolic pathway that enable the synthesis of proline from glutamate. Furthermore, in E. coli grown under reduced temperature (10° C.) it is known that proline isomerization enables cold-shock adaptation, often requiring the assistance of a peptidyl-prolyl cis-trans-isomerase for correct folding of proteins, which again points to a key role of proline in cold adaptation^{25,26}. The novel and unexpected technical effects of the current disclosure support a conclusion that forcing E. coli to grow under proline-deficient conditions caused activation of cold-stress pathways or promote the production of PUFAs through the exogenous PUFA synthase pathway. Consistent with this, the current disclosure demonstrates the unexpected technical result of PUFA production being E. coli strain dependent an important factor in scaling for industrial use.

### SUMMARY

In summary, the current disclosure demonstrates the unexpected technical effect that the JM109 strain harboring pEPAΔ1,3,4,9 in conjunction with pfaR results in a five-fold increase in total FA when compared with the controls and a near two-fold change when compared to pET-pEPA Δ1,3,4,9. The increase is mainly driven by monosaturated FAs and to a lesser extent by saturated and ω6 FAs pointing to a regulatory difference in the PUFA synthase pathway dependent on the presence or absence of pfaR, a novel and unexpected finding. Further, although this strain produced ω3-FA at yields comparable to the already known pET-pEPA A1,3,4,9 strain, its profile of ω3-FAs produced lower yields of EPA; higher yields of 8,11,14,17-eicosatetraenoic acid, a known intermediate in the synthesis of EPA; and higher yields of 7, 10, 13, 16, 19-docosapentaenoic acid, a known intermediate in the synthesis of DHA. Further the strain harboring pEPA A1,3,4,9 as well as the strain harboring pEPA A1,3,4,9 in conjunction with pfaR, showed altered ratios of saturated to monounsaturated FAs.

An additional unexpected result is the demonstration herein that S. oneidensis pfaR enhanced production of the typical endogenous monounsaturated fatty acids from E. coli, namely 18:1 and 22:1.

The unexpected high yields of monounsaturated FAs (MUFAs) demonstrated herein can be harnessed for commercial development. The biodiesel industry regards MUFAs as ideal components in fuel formulations due to their fluidity at low temperatures and their oxidative stability¹⁷. However, PUFA's are undesirable in biodiesel preparations due to their propensity to oxidize. The current disclosure further demonstrates that fatty acid mixtures obtained from both the pET-pEPAΔ1, 3, 4, 9 strains and pfaR-pEPAΔ1, 3, 4, 9 strains have ratios of saturated: monounsaturated:ω3:ω6 FAs that are comparable to the ratios observed in the fish sources that are considered to be good sources of FAs, such as copper river salmon ³⁰.

The results and examples herein demonstrate that overexpression of pfaR alters the fatty acid profile of E. coli JM109 carrying the minimum set of genes for ω3-FA production, causing a significant increase in the total FA yield by tripling the yields of monounsaturated FAs and doubling the yields of saturated FAs. Also demonstrated is accumulation of the 20:4 precursor to eicosapentaenoic acid (EPA, 20:5) with a concomitant decrease in EPA yield. These changes in FA production required both pfaR and the other PUFA synthase genes, indicating that the product of pfaR causes these changes through the regulation of the PUFA synthase pathway

More specifically, co-expression of pfaR together with five pfa genes deemed essential for PUFA production (contained in plasmid pEPAΔ1,3,4,9), resulted in an increase in the total fatty acid yield, driven mainly by an increase in monosaturated FAs and saturated FAs. The recombinant E. coli harboring pfaR showed a decrease in EPA production with a concomitant increase in eicosatetraenoic acid (ETA; 20:4ω3), a known precursor of EPA. This work discloses alteration of genes in the PUFA synthase cluster results in the production of different FA profiles in bacteria with a variety of possible industrial applications.

Having described the invention in detail and by reference to specific aspects and/or embodiments thereof, it will be apparent that modifications and variations are possible without departing from the scope of the invention defined in the appended claims. More specifically, although some aspects of the present invention can be identified herein as particularly advantageous, it is contemplated that the present invention is not limited to these particular aspects of the invention. Percentages disclosed herein can vary in amount by ±10, 20, or 30% from values disclosed and remain within the scope of the contemplated invention.

TABLE 5
Sequence Table

| | SEQ ID | |
|---|---|---|
| Name | NO | Sequence |
| pfasRSO | 1 | MSSPKVGTQVADIVRKQKSSTEKNSRHKVVRHRN |
| Protein | | ATTTPEMRHFIQQSELSVSQLAKILNITEATVRK |
| | | WRKRESINDSPNTPHHLNTTLTPIEEYVVVGLRY |
| | | QLKLPLDRLLKATQTFINPNVSRSGLARCLKRYG |
| | | VSRLDEFDQELVPKQYFNQLPIAQGSDVQTYTVN |
| | | SETLAKALALPSTDGDTVVQVVSLTLPPQLTEAK |
| | | PKSVLLGIDSKTDWIYIDIYQDSNTQAANRYIAY |
| | | VLQDGPFHLRKLLVRNYHTFLARFPGVQTPKAIA |
| | | KSLNKATATRFASGDS |
| pfasRSO | 2 | TTATGAGTCTCCACTGGCAAAGCGGGTGGCTGTT |
| | | GCCTTATTGeneAAGGGATTTAGCGATGGCTTTT |
| | | GGGGTTTGCACGCCAGGAAAGCGGGCTAAAAATG |
| | | TGTGGTAGTTGCGCACCAGCAATTTACGCAGATG |
| | | AAACGGCCCGTCTTGCAGTACATAAGCGATATAG |
| | | CGATTGGCCGCTTGGGTATTACTGTCTTGATAAA |
| | | TATCGATATAAATCCAATCTGTCTTGCTATCTAT |
| | | ACCAAGGAGCACTGATTTAGGCTTGGCTTCGGTT |
| | | AGTTGCGGTGGCAGTGTTAATGACACCACTTGCA |
| | | CTACGGTGTCGCCATCGGTGCTTGGCAGGGCGAG |
| | | CGCTTTTGCTAAGGTTTCGGAGTTAACCGTATAG |
| | | GTTTGTACATCGCTGCCTTGGGCAATCGGTAATT |
| | | GATTAAAATACTGTTTAGGGACAAGCTCTTGGTC |
| | | AAACTCGTCGAGGCGCGATACCCCATAACGCTTA |
| | | AGGCAACGGGCTAAGCCTGAGCGCGATACATTGG |
| | | GGTTAATAAAGGTTTGCGTGGCTTTAAGTAGCCT |
| | | ATCGAGCGGCAGCTTTAGCTGATACCTCAGTCCC |
| | | ACCACCACATATTCTTCAATTGGGGTGAGGGTGG |
| | | TATTGAGATGATGCGGTGTATTCGGGCTATCGTT |
| | | AATCGATTCGCGCTTACGCCATTTACGCACTGTG |
| | | GCTTCGGTAATATTTAAAATTTTAGCAAGCTGAC |
| | | TGACACTCAATTCAGATTGCTGAATAAAGTGGCG |
| | | CATTTCTGGCGTGGTGGTGGCATTTCGGTGTCTT |
| | | ACCACCTTGTGGCGAGAGTTTTTCTCTGTGCTCG |
| | | ATTTTTGTTTGCGAACAATATCTGCGACTTGAGT |
| | | GCCAACTTTTGGAGAGCTCAT |

### REFERENCES

1. Metz, J. G. et al. Production of Polyunsaturated Fatty Acids by Polyketide Synthases in Both Prokaryotes and Eukaryotes. Science (80-.). 293, 290-293 (2001).
2. Yazawa, K., Yamada, A., Kato, S. & Kondo, K. U.S. Pat. No. 568,3898A. (1997).
3. Campbell, M. Patent Application Publication (10) Pub. No.: US 2015/0176237 A1. vol. 1 3-7 (2015).
4. Yoshida, K. et al. Bacterial long-chain polyunsaturated fatty acids: Their biosynthetic genes, functions, and practical use. Mar. Drugs 14, 94 (2016).
5. Yazawa, K. Production of eicosapentaenoic acid from marine bacteria. Lipids 31 Suppl, S297-300 (1996).
6. Orikasa, Y. et al. Characterization of the eicosapentaenoic acid biosynthesis gene cluster from Shewanella sp strain SCRC-2738. Cell. Mol. Biol. 50, 625-630 (2004).
7. Okuyama, H., Orikasa, Y., Nishida, T., Watanabe, K. & Morita, N. Bacterial genes responsible for the biosynthesis of eicosapentaenoic and docosahexaenoic acids and their heterologous expression. Appl. Environ. Microbiol. 73, 665-670 (2007).
8. Orikasa, Y. et al. PfaB products determine the molecular species produced in bacterial polyunsaturated fatty acid biosynthesis. FEMS Microbiol. Lett. (2009) doi: 10.1111/j.1574-6968.2009.01582.x.
9. Hayashi, S. et al. Control Mechanism for cis Double-Bond Formation by Polyunsaturated Fatty-Acid Synthases. Angew. Chemie-Int. Ed. 58, 2326-2330 (2019).
10. Kram, K. E. & Finkel, S. E. Culture volume and vessel affect long-term survival, mutation frequency, and oxidative stress of Escherichia coli. Appl. Environ. Microbiol. (2014) doi: 10.1128/AEM.03150-13.
11. Moi, I. M. et al. Polyunsaturated fatty acids in marine bacteria and strategies to enhance their production. Appl. Microbiol. Biotechnol. 102, 5811-5826 (2018).
12. Yu, R. et al. Production of eicosapentaenoic acid by a recombinant marine cyanobacterium, Synechococcus sp. Lipids (2000) doi: 10.1007/s11745-000-0619-6.
13. Yazawa, K. et al. Eicosapentaenoic Acid Productivity of the Bacteria Isolated from Fish Intestines. Nippon Suisan Gakkaishi (Japanese Edition) vol. 54 1835-1838 (1988).
14. Giner-Robles, L., Lázaro, B., de la Cruz, F. & Moncalián, G. fabH deletion increases DHA production in Escherichia coli expressing Pfa genes. Microb. Cell Fact. (2018) doi: 10.1186/s12934-018-0928-5.
15. Bianchi, A. C., Olazábal, L., Torre, A. & Loperena, L. Antarctic microorganisms as source of the omega-3 polyunsaturated fatty acids. World J. Microbiol. Biotechnol. 30, 1869-1878 (2014).
16. Gong, Y. et al. Metabolic engineering of microorganisms to produce omega-3 very long-chain polyunsaturated fatty acids. Prog. Lipid Res. 56, 19-35 (2014).
17. Cao, Y. et al. Production of free monounsaturated fatty acids by metabolically engineered Escherichia coli. Biotechnol. Biofuels (2014) doi: 10.1186/1754-6834-7-59.
18. Amoabediny, G. & Buchs, J. Modelling and advanced understanding of unsteady-state gas transfer in shaking bioreactors. Biotechnol. Appl. Biochem. (2007) doi: 10.1042/ba20060120.
19. Crusemann, M. et al. Prioritizing Natural Product Diversity in a Collection of 146 Bacterial Strains Based on Growth and Extraction Protocols. Journal of Natural Products (2017) doi: 10.1021/acs.jnatprod.6b00722.
20. Fichman, Y. et al. Evolution of proline biosynthesis: Enzymology, bioinformatics, genetics, and transcriptional regulation. Biol. Rev. (2015) doi: 10.1111/brv. 12146.
21. Yanisch-Perron, C., Vieira, J. & Messing, J. Improved M13 phage cloning vectors and host strains: nucleotide sequences of the M13mpl8 and pUC 19 vectors. Gene (1985) doi: 10.1016/0378-1119 (85) 90120-9.
22. Raymond-Bouchard, I. et al. Conserved genomic and amino acid traits of cold adaptation in subzero-growing Arctic permafrost bacteria. FEMS Microbiol. Ecol. (2018) doi:10.1093/femsec/fiy023.
23. M K, C. Mechanism of bacterial adaptation to low temperature. J. Biosci. 31, 157-65. (2006).
24. Zhao, J. S., Deng, Y., Manno, D. & Hawari, J. Shewanella spp. genomic evolution for a cold marine lifestyle and in-situ explosive biodegradation. PLOS One (2010) doi: 10.1371/journal.pone.0009109.
25. Kandror, O. & Goldberg, A. L. Trigger factor is induced upon cold shock and enhances viability of Escherichia coli at low temperatures. Proc. Natl. Acad. Sci. U.S.A. (1997) doi: 10.1073/pnas.94.10.4978.
26. Budiman, C., Koga, Y., Takano, K. & Kanaya, S. FK506-binding protein 22 from a psychrophilic bacterium, a cold shock-inducible peptidyl prolyl isomerase with the ability to assist in protein folding. Int. J. Mol. Sci. 12, 5261-5284 (2011).
27. Rock, C. O. & Jackowski, S. Forty years of bacterial fatty acid synthesis. Biochem. Biophys. Res. Commun. 292, 1155-1166 (2002).
28. Lu, Y. J., Zhang, Y. M. & Rock, C. O. Product diversity and regulation of type II fatty acid synthases. Biochem. Cell Biol. 82, 145-155 (2004).
29. Oyola-Robles, D., Rullan-Lind, C., Carballeira, N. M. & Baerga-Ortiz, A. Expression of dehydratase domains from a polyunsaturated fatty acid synthase increases the production of fatty acids in Escherichia coli. Enzyme Microb. Technol. 55, 133-139(2014).
30. Weaver, K. L. et al. The Content of Favorable and Unfavorable Polyunsaturated Fatty Acids Found in Commonly Eaten Fish. J. Am. Diet. Assoc. (2008) doi: 10.1016/j.jada.2008.04.023.
31. Davies, A. N. The new Automated Mass Spectrometry Deconvolution and Identification System (AMDIS). Int. J. Ion Mobil. Spectrom. 4, 165-172 (2001).
32. Dodds, E. D., McCoy, M. R., Rea, L. D. & Kennish, J. M. Gas chromatographic quantification of fatty acid methyl esters: Flame ionization detection vs. electron impact mass spectrometry. Lipids 40, 419-428 (2005).
33. Takeyama, H., Takeda, D., Yazawa, K., Yamada, A. & Matsunaga, T. Expression of the eicosapentaenoic acid synthesis gene cluster from Shewanella sp. In a transgenic marine cyanobacterium, Synechococcus sp. Microbiology (1997) doi: 10.1099/00221287-143-8-2725.
34. Lee, S. J. S. J., Jeong, Y. S. Y. S., Kim, D. K. D. U., Seo, J. W. J. W. & Hur, B. K. B. K. Eicosapentaenoic acid (EPA) biosynthetic gene cluster of Shewanella oneidensis MR1. Biotechnol. Bioprocess Eng. 11, 510-516 (2006).
35. Lee, W. H. et al. Identification of psychrophile Shewanella sp. KMG427 as an eicosapentaenoic acid producer. J. Microbiol. Biotechnol. (2008) doi: 10.4014/jmb.0800.102.
36. Orikasa, Y. et al. Recombinant production of docosahexaenoic acid in a polyketide biosynthesis mode in Escherichia coli. Biotechnol. Lett. (2006) doi: 10.1007/s10529-006-9168-6.
37. Amiri-Jami, M. & Griffiths, M. W. Recombinant production of omega-3 fatty acids in Escherichia coli using a gene cluster isolated from Shewanella baltica MAC1. J. Appl. Microbiol. (2010) doi: 10.1111/j.1365-2672.2010.04817.x.
38. Amiri-Jami, M., Abdelhamid, A. G., Hazaa, M., Kakuda, Y. & Griffths, M. W. Recombinant production of omega-3 fatty acids by probiotic Escherichia coli Nissle 1917. FEMS Microbiol. Lett. (2015) doi: 10.1093/femsle/fnv166.
39. Peng, Y. F. et al. DHa production in Escherichia coli by expressing reconstituted key genes of polyketide synthase pathway from marine bacteria. PLOS One (2016) doi: 10.1371/journal.pone.0162861

What is claimed is:

1. An in vitro method of producing fatty acids, wherein an E. coli containing pfaA, pfaB, pfaC, pfaD and/or pfaE genes (pEPAΔ1, 3, 4, 9) overexpresses gene regulator pfaR.

2. The method of claim 1, wherein the E. coli strain is JM109.

3. The method of claim 1, wherein the E. coli strain is DH10B.

4. The method of clam 1, wherein the E. coli is incubated at 15° C.

5. The method of claim 1, wherein the E. coli are grown in cell culture flasks, tubes, or bags containing media and nutrients equivalent to twenty percent of the total volume.

7. The method of claim 1, wherein E. coli are grown in Laura Broth without carbon supplementation.

8. The method of claim 1, wherein endogenous acyl-CoA synthetase structural gene, fadD is deleted.

9. The method of claim 2, wherein saturated, monosaturated and polyunsaturated fatty acid synthesis is increased.

10. The method of claim 9, wherein the increased polyunsaturated fatty acid increase includes omega-3 three and omega 6 fatty acids.

11. A pfaRso plasmid, wherein the gene sequence of pfaRso gene has greater than 90% homology to SEQ ID NO. 1.

12. The plasmid of claim 11, wherein the plasmid is grown under anaerobic conditions.

13. The plasmid of claim 11, wherein the plasmid contains pfaA, pfaB, pfaC, pfaD and/or pfaE genes (pEPAΔ1, 3, 4, 9)
